(19) United States
(12) Patent Application Publication
(10) Pub. No.: US 2007/0203065 A1
(43) Pub. Date: Aug. 30, 2007

(54) Insulin-Independent, Bone Morphogenetic Protein (Bmp)-Mediated Uptake Of Blood Glucose By Peripheral Cells And Tissues

(75) Inventors: Slobodan Vukicevic, Zagreb (HR); Petra Simic, Zagreb (HR)

Correspondence Address: Leon R Yankwich;Yankwich & Associates, 201 Broadway, Cambridge, MA, 02139, US

(73) Assignee: GENERA DOO, Krapinske Toplice (HR)

(21) Appl. No.: 11/628,150

(22) PCT Filed: Jun. 2, 2005
(86) PCT No.: PCT/US05/19302

§ 371 Date: Nov. 29, 2006

Related U.S. Application Data
(60) Provisional application No. 60/576,860, filed on Jun. 3, 2004.
(60) Provisional application No. 60/608,798, filed on Sep. 10, 2004.

(51) Int. Cl. A61K 38/18

(52) U.S. Cl. 514/12

(57) ABSTRACT

Methods and compositions comprising a bone morphogenetic protein (BMP) are described for stimulating uptake of serum glucose by peripheral cells and tissues, for treating type 1 or type 2 diabetes, for controlling exocrine pancreatic function, and for treating pancreatitis by an insulin-independent pathway.

### CROSS-REFERENCE TO RELATED APPLICATIONS

This application claims priority to U.S. Provisional Application No. 60/576,860, filed Jun. 3, 2004, and U.S. Provisional Application No. 60/608,798, filed Sep. 10, 2004.

### GENERAL FIELD OF THE INVENTION

This invention is generally in the field of regulation of glucose metabolism in the mammalian body. In particular, the invention provides compositions and methods for enhancing uptake of glucose from the blood by peripheral cells and tissues by administrating to an individual a bone morphogenetic protein (BMP).

### BACKGROUND OF THE INVENTION

Glucose is among the most fundamental of sources of carbon and energy for cells. The regulation of the level of blood glucose circulating throughout the body of an individual is critical for maintaining proper metabolic stasis and overall health. The need to properly maintain serum glucose levels (glucose homeostasis) is no better illustrated than in the chronic disease diabetes mellitus (diabetes). In diabetes the body loses the ability to properly produce or respond to the hormone insulin so that cells of the peripheral tissues fail to actively take up glucose from the blood for use or storage. In the diabetic individual, the level of glucose in the peripheral blood can become elevated (hyperglycemia) and typically remains so unless some form of intervention is employed (e.g., administration of exogenous insulin) to return glucose in the blood to normal levels. Left unchecked, the hyperglycemia of diabetic individuals can result in shock, organ degeneration or failure (e.g., kidney failure, blindness, nerve disease, cardiovascular disease), tissue necrosis (e.g., requiring foot amputation), and even death.

Two major forms of diabetes are type 1 and type 2 diabetes. Type 1 diabetes, which was previously known as insulin-dependent diabetes mellitus (IDDM) or juvenile onset diabetes, is an autoimmune disease in which the body destroys the insulin-producing P cells (islet cells) of the pancreas resulting in an absolute requirement for daily administration of exogenous insulin to maintain normal blood glucose levels. Type 1 diabetes usually is diagnosed in children and young adults, but can occur at any age. Type 1 diabetes accounts for 5-10% of diagnosed cases of diabetes.

By far the most prevalent form of diabetes is type 2 diabetes, which was previously known as non-insulin-dependent diabetes mellitus (NIDDM). Type 2 diabetes was also previously known as adult-onset diabetes, however, this form of diabetes is becoming increasingly prevalent in the growing population of overweight and clinically obese children and young adults. Type 2 diabetes accounts for approximately 90-95% of all diagnosed cases of diabetes. Type 2 diabetes typically begins with insulin resistance, a disorder in which the body's cells do not respond to insulin properly, followed by a gradual loss on part of the pancreas to produce and secrete insulin. Type 2 diabetes is associated with a variety of factors including older age, obesity, family history of diabetes, history of gestational diabetes, impaired glucose metabolism, physical inactivity, and various races or ethnicities. Individuals with type 2 diabetes must attempt to control their blood glucose level with careful diet, exercise and weight reduction, and additional medications.

Administering exogenous insulin (e.g., by pump or injection) has been the standard method of treating type 1 diabetes, although treatments for type 2 diabetes may also include insulin supplementation. A number of drugs have also been developed that may be employed in various regimens to treat diabetes. Such drugs include metformin that enhances the action of insulin in the liver, sulfonylureas that enhance insulin production and secretion by the pancreas, biguanides that decrease the amount of glucose made by the liver, thiazolidinediones that enhance the sensitivity of peripheral tissues to the action of insulin, meglinitides that stimulate insulin production, and D-phenylalanine that stimulates the rate of insulin production.

Cases of diabetes are expected to have increased between the period of 1995 and 2010 by 35% in the United States and by 87% worldwide (Zimmet, J. Intern. Med., 247: 301-310 (2000)).

Clearly, needs remain for the effective regulation of proper blood glucose levels, not only to improve treatments for diabetes, but potentially other conditions in which the body benefits by improved efficiency in uptake of serum glucose by the peripheral tissues.

### SUMMARY OF THE INVENTION

The invention addresses the above problems by providing means and methods for regulating the level of glucose in the blood of humans and other mammals through an insulin-independent pathway. In particular, the invention is based on the discovery that a bone morphogenetic protein (“BMP”, “morphogen”), such as BMP-6 or BMP-7, is able to effectively promote uptake of blood glucose (“serum glucose”) by peripheral tissues and cells of an individual by an insulin-independent pathway. Thus, the methods of the invention are effective for treating hyperglycemia by an insulin-independent pathway in both healthy and diabetic individuals and also for maintaining healthy blood glucose levels even in cases of severe diabetes.

In one embodiment, the invention provides a method of enhancing or stimulating uptake of blood glucose by peripheral cells and tissues in an individual by an insulin-independent pathway comprising administering to the individual an effective amount of aBMP.

In another embodiment, the invention provides a method of treating a hyperglycemic condition in an individual comprising administering to the individual an effective amount of a BMP.

In yet another embodiment, the invention provides a method of treating diabetes in an individual comprising administering to the individual an effective amount of a BMP, such as BMP-6, BMP-7, or heterodimer thereof. Since BMP-mediated regulation of blood glucose levels proceeds by an insulin-independent pathway, this method of treatment is applicable to both type 1 and type 2 diabetes.

In another embodiment, the invention provides a method of modulating or controlling exocrine pancreatic fimction in an individual comprising administering to the individual an effective amount of a BMP.

In yet another embodiment, the invention provides a method of reducing the level of amylase in the blood of an individual comprising administering to the individual an effective amount of a BMP.

In still another embodiment, the invention provides a method of treating acute and chronic forms of pancreatitis in an individual comprising administering to the individual an effective amount of a BMP.

Methods and compositions described herein may also be used in combination with insulin and/or any of a variety of other compounds that are used to treat diabetes, hyperglycemia, or pancreatitis.

In another embodiment, the invention provides methods of identifying candidate compounds for use in stimulating uptake of blood glucose by peripheral cells and tissues, for treating hyperglycemia, and/or for treating diabetes. A particularly preferred method of identifying such a candidate compound may comprise the steps of:
incubating a culture of pancreatic β-cells or hepatocytes in the presence and absence of a test compound, wherein the pancreatic β-cells or hepatocytes comprise functional genetic information necessary for synthesis of a BMP,
assaying the cells for the level of synthesis of the BMP,
comparing the level of synthesis of BMP in the presence and absence of the test compound, wherein a higher level of BMP synthesis in the presence than in the absence of the test compound indicates that the test compound is a candidate compound for treating hyperglycemia or diabetes.

A candidate compound identified by a method as described above may also be tested in vivo for the ability to decrease the level glucose in the peripheral blood of a mammal, including any of a variety of animal models employed for studying diabetes.

Particularly useful in the compositions and methods of the invention is a BMP selected from the group consisting of BMP-6, BMP-7, and heterodimers thereof.

### BRIEF DESCRIPTION OF THE DRAWINGS

FIGS. 1A and 1B show micrographs of sections of pancreatic tissue from wild type and BMP-6 knock-out mice, respectively, immunostained using an anti-insulin antibody to identify insulin producing β-cells of the islands of Langerhans as described in Example 1. Pancreatic tissue from BMP-6 knock-out mice (FIG. 1B) had significantly (P<0.05) fewer number of insulin positive cells compared to tissue from wild type mice (FIG. 1A). See text for details.

FIGS. 2A and 2B show micrographs of sections of liver tissue from wild type and BMP-6 knock-out mice, respectively, immunostained with anti-glucagon antibody as describe in Example 1. Liver tissue from BMP-6 knock-out mice (FIG. 2B) had significantly fewer number of glucagon positive cells compared to tissue from wild type mice (FIG. 2A). See text for details.

FIG. 3 shows bar graphs that indicate the concentration of insulin (μg/L) in blood samples drawn from BMP-6 knock-out mice (n=20) that did not receive BMP-6 (knock-out control, “KO-C”) and in serum samples drawn from BMP-6 knock-out mice (n=20) 1 hour after receiving an intravenous (i.v.) injection of BMP-6 (10 μg/kg of body weight, “KO+BMP”) as described in Example 2. The level of insulin in sera from BMP-6 knock-out animals treated with BMP-6 was significantly higher (P<0.01) than the level in sera from control animals. See text for details.

FIG. 4 shows bar graphs that indicate the concentration of insulin (μg/L) in blood samples drawn from wild type mice (n=20) that did not receive BMP-6 (wild type control, “WT-C”) and in serum samples drawn from wild type mice (n=20) 1 hour after receiving an i.v. injection of BMP-6 (10 μg/kg, “WT+BMP”) as described in Example 2. The level of insulin in sera from wild type mice treated with BMP-6 was not significantly higher from the level in sera from control animals. See text for details.

FIG. 5 shows bar graphs that indicate the glucose levels (mmol) in blood samples drawn from wild type mice that received no BMP-6 (control) and from wild type mice that received an i.v. injection of (mature) BMP-6 (20 μg/kg). Glucose (650 mg/kg) was orally administered to all of the animals and serum samples were taken 6 hours following injection of BMP-6 according to the protocol described in Example 3. See text for details.

FIG. 6 shows bar graphs that indicate the glucose levels (mmol) in blood samples drawn from BMP-6 knock-out mice that received no BMP-6 (control) and from BMP-6 knock-out mice that received an i.v. injection of (mature) BMP-6 (20 μg/kg). Glucose was orally administered to all of the animals and serum samples were taken at 2 hours and 24 hours after administration of BMP-6 according to the protocol described in Example 3. The levels of glucose in sera from the BMP-6 treated mice were significantly lower than those of control animals at 2 hours (P<0.009) as well as at 24 hours (P<0.02) following injection of BMP-6. See text for details.

FIG. 7 shows bar graphs that indicate the glucose levels (mmol) in blood samples drawn from rats that received no BMP-6 (control) and from rats that received i.v. injection of (mature) BMP-7 (100 μg/kg). Glucose was orally administered to all of the animals and serum samples were taken at 0, 45 minutes, 2 hours, 4 hours, and 6 hours following injection of BMP-7 according to the protocol described in Example 3. The levels of glucose in sera from BMP-7 treated rats were significantly lower than those of untreated rats at 45 minutes (P<0.006) and 2 hours (P<0.004) after administration of BMP-7. See text for details.

FIG. 8 shows bar graphs that indicate the glucose levels (mmol) in blood samples drawn from rats that received no BMP-7 (control) and from rats that received i.v. injection of soluble (i.e., unprocessed) BMP-7 (sBMP, 60 μg/kg). Glucose was orally administered to all of the animals and serum samples taken at 0, 45 minutes, 2 hours, 4 hours, and 26 hours following injection of sBMP according to the protocol described in Example 3. The levels of glucose in sera from the sBMP-7 treated rats were significantly lower than those of untreated rats at 2 hours (P<0.004), 4 hours (P<0.05), and 26 hours (P<0.05) after administration of sBMP-7. See text for details.

FIG. 9 shows bar graphs that indicate the glucose levels (mmol) in blood samples drawn from rats at 2 hours following administration of various doses of (mature) BMP-7 or sBMP-7 according to the protocol described in Example 3. The levels of glucose in the sera from most of the animals treated with either form of BMP-7 were significantly lower than that of control animals (no BMP-7 treatment) as indicated by the various P values above the bars. See text for details.

FIG. 10 shows bar graphs that indicate the levels of amylase (units per liter; “units/L”) in blood samples drawn from wild type mice that received no BMP-6 (control) and that received BMP-6 at a dose of 5 μg/kg or 20 μg/kg. Glucose was orally administered to all animals and serum samples taken 6 hours following injection of BMP-6 according to the protocol described in Example 3. The levels of amylase in sera from the BMP-6 treated animals were significantly lower than those of control animals as indicated by the P values above the bars. See text for details.

FIG. 11 shows bar graphs that indicate the levels of amylase (U/L) in blood samples drawn from BMP-6 knock-out mice that received no BMP-6 (control) and knock-out mice that received BMP-6 at a dose of 20 μg/kg. Glucose was orally administered to all animals and serum samples taken at 6 hours, 16 hours, and 24 hours following injection of BMP-6 according to the protocol described in Example 3. The levels of amylase in sera from the BMP-6 treated animals were significantly lower than those of control animals as indicated by the P values above the bars. See text for details.

FIG. 12 shows bar graphs that indicate the levels of amylase in blood samples drawn from rats that did not receive BMP-6 (control) and from rats that received i.v. injection of BMP-6 (5 μg/kg). Glucose was orally administered to all of the animals and serum samples were taken at 0, 45 minutes, 2 hours, 4 hours, 6 hours, and 26 hours following injection of BMP-6 according to the protocol described in Example 3. The levels of amylase in sera from the BMP-6 treated rats were significantly lower than those of untreated rats at most of the time points as illustrated by selected P values above the bars. See text for details.

FIG. 13 shows bar graphs that indicate the levels of amylase in blood samples drawn from rats 45 minutes following i.v. injection of BMP-6 (5 μg/kg), (mature) BMP-7 (100 μg/kg), and various doses of sBMP-7 according to the protocol described in Example 3. The levels of amylase in the sera from all of the animals treated with BMP-6, BMP-7, or sBMP-7 were significantly lower than that of control animals (no BMP-6 or BMP-7 treatment) as indicated by the various P values above the bars. See text for details.

FIG. 14 shows a graph of ¹⁸fluoro-deoxyglucose (¹⁸FDG) in counts per minute (cpm) as a function of time (minutes) in the blood of rats that received an intravenous (i.v.) injection of ¹⁸FDG (animal 1, diamonds), an i.v. injection of BMP-6 (60 μg/kg) and an injection of ¹⁸FDG (animal 2, squares) essentially at the same time, or an injection of BMP-6 at 2 hours prior to administration of ¹⁸FDG (animal 3, triangles) as described in Example 4. See text for details.

FIG. 15 shows a graph of ¹⁸fluoro-deoxyglucose (¹⁸FDG) in counts per minute (cpm) as a function of time (minutes) in the blood of rats that were treated with alloxan (75 mg/kg) to induce diabetes followed by an intravenous (i.v.) injection of ¹⁸FDG (“Diabetes”, triangles) or followed by an i.v.injection of ¹⁸FDG and an i.v. injection of BMP-6 (60 μg/kg) at essentially the same time (“Diabetes+BMP-6”) according to the protocol described in Example 5. See text for details.

FIG. 16 shows bar graphs that indicate the level of ¹⁸fluoro-deoxyglucose (¹⁸FDG) in counts per minute (cpm×10⁵) in the urine of rats that were treated with alloxan to induce diabetes followed by an intravenous (i.v.) injection ¹⁸FDG (“DIABETES”) or followed by an i.v.injection of ¹⁸FDG and an i.v. injection of BMP-6 (60 μg/kg) at essentially the same time (“DIABETES+BMP-6”) according to the protocol described in Example 5. Urine was collected 3 hours after injection of ¹⁸FDG. See text for details.

FIG. 17 shows a graph of blood glucose levels (mmol) as a function of time (hours) in severely diabetic (nonobese diabetic, “NOD”) mice that require insulin intravenous (i.v.) injections every 12 hours to avoid dying of severe hyperglycemia. Levels of glucose were determined in samples of blood obtained from NOD mice (n=2) that received insulin, i.v., every 12 hours (squares) and from NOD mice (n=6) that received no insulin, but instead received a single i.v. injection BMP-6 (60 μg/kg, triangles) according to the protocol described in Example 6. Both NOD mice that received insulin eventually died within 30 hours. See text for details.

FIG. 18 shows a graph of the percent (%) survival of severely diabetic NOD mice described above for FIG. 17 over time (hours). Both of the NOD mice receiving insulin every 12 hours eventually died within 30 hours (squares), whereas 5 of the 6 NOD mice that received a single i.v. injection of BMP-6 and no insulin (triangles) survived the course of the experiment. See text of Example 6 for details.

FIG. 19 shows bar graphs of the fold-change over time in the level of expression of the enzyme PEPCK involved in gluconeogenesis in the liver of NOD mice that received a single i.v. injection of BMP-6 (60 μg/kg) compared to the level of expression in the NOD mice 6 hours after receiving BMP-6 (bar at 6 hours is 1-fold change) according to the protocol in Example 7. See text for details.

FIG. 20 shows bar graphs of the fold-change over time in the level of expression of the mitochondrial transcription factor PGC1α involved in expression of oxidative enzymes in the liver of NOD mice that received a single i.v. injection of BMP-6 (60 μg/kg) compared to the level of expression in the NOD mice prior to receiving BMP-6 (bar at 0 hours is 1-fold change) as described in the protocol in Example 7. See text for details.

FIG. 21 shows a graph of blood glucose levels (mmol) as a function of time (minutes) in rats that received no treatment (“CONTROL”, diamonds), BMP-6 (60 μg/kg, i.v.) (“BMP-6”, squares), or the endoprotease furin (10 μL/kg) (“FURIN”) according to the protocol described in Example 8. See text for details.

FIG. 22 shows a graph of blood glucose levels (mmol) as a function of time (minutes) in rats that received glucose (2 g/kg, i.v.) (“GLUCOSE”, diamonds), glucose and BMP-6 (60 μg/kg, i.v.) (“GLUCOSE+BMP-6”, squares), or glucose and furin (10 μL/kg) (“GLUCOSE+FURIN”, triangles) according to the protocol described in Example 8. See text for details.

FIG. 23 shows bar graphs that indicate the glucose levels (mmol) in blood drawn from rats 15 minutes after receiving glucose (2 g/kg, i.v.) (“GLUCOSE”), glucose and furin (10 μL/kg) (“GLUCOSE+FURIN”), or glucose and BMP-6 (60 μg/kg, i.v.) (“GLUCOSE+BMP-6”) according to the protocol described in Example 8. See text for details.

FIG. 24 shows bar graphs that indicate the glucose levels (mmol) in blood drawn from rats 30 minutes after receiving glucose (2 g/kg, i.v.) (“GLUCOSE”), glucose and BMP-6 (60 μg/kg, i.v.) (“GLUCOSE+BMP-6”), glucose and furin (10 μL/kg) (“GLUCOSE+FURIN”), or glucose, furin, and anti-BMP polyclonal antibody (“GLUCOSE+FURIN+anti-BMP”) according to the protocol described in Example 9. See text for details.

FIG. 25 shows a Western immunoblot of samples rat plasma treated according to the protocol described in Example 10 and electrophoresed on a polyacrylamide under reducing (+DTT) and non-reducing (−DTT) conditions to detect monomer and dimer forms of BMP-7, respectively. Lanes 1 (−DTT) and 2 (+DTT) contain BMP-7 standard. Lanes 3 (+DTT) and 4 (−DTT) contain plasma spiked with BMP-7 standard. Lanes 5 (+DTT) and 6 (−DTT) contain plasma treated with furin. Horizontal arrows indicate the relative positions of 35 kilodalton (kDa) (mature BMP dimer, lane 6) and 17 kDa molecular weight protein species.

### DETAILED DESCRIPTION OF THE INVENTION

This invention is based on the discovery that administration of a bone morphogentic protein (BMP), such as BMP-6 or BMP-7, to an individual (a mammal) is effective to enhance or stimulate uptake of glucose in the circulating blood by peripheral cells and tissues of the individual. Moreover, the ability of such BMPs to regulate (i.e., reduce) serum glucose levels occurs via an insulin-independent pathway. Consistent with this discovery is the finding that such BMPs reduce expression of key liver enzymes involved in gluconeogenesis and activate expression of lipid metabolism enzymes. Accordingly, the invention provides means and methods for regulating, i.e., stimulating, glucose uptake by the peripheral cells and tissues of an individual, preventing or correcting undesirable hyperglycemic conditions, and also for treating diabetes comprising administering to an individual an effective amount of a BMP, such as BMP-6, BMP-7, or heterodimers thereof. Moreover, it is appreciated that the ability of BMPs, such as BMP-6 and BMP-7, to stimulate uptake of blood glucose by peripheral cells improves the capacity of such cells to survive stressful and potentially destructive conditions, such as may result from physical exercise or exertion, various metabolic disorders, and physical trauma, including various medical procedures.

In order that the invention may be more clearly understood, the following terms are defined below.

An “individual” or “patient” is a human or other mammal that has, is suspected to have, or is being diagnosed for a hyperglycemic condition or diabetes.

A “bone morphogenetic protein” (also referred to as “BMP” or “morphogen”) is any member of a particular subclass of the transforming growth factor-β (TGF-β) super family of proteins (see, e.g., Hoffmann et al., Appl. Microbiol. Biotechnol., 57: 294-308 (2001); Reddi, J. Bone Joint Surg, 83-A(Supp. 1): S1-S6 (2001); U.S. Pat. Nos. 4,968,590; 5,011,691; 5,674,844; and 6,333,312). All BMPs have a signal peptide, prodomain, and a carboxy terminal (mature) domain. The carboxy terminal domain is the mature form of the BMP monomer and contains a highly conserved region characterized by seven cysteine residues that form a cysteine knot (see, Griffith et al., Proc. Natl. Acad. Sci. USA, 93: 878-883 (1996)).

BMPs were originally isolated from mammalian bone using protein purification methods (see, e.g., Urist et al., Proc. Soc. Exp. Biol. Med., 173: 194-199 (1983); Urist et al., Proc. Natl. Acad. Sci. USA, 81: 371-375 (1984); Sampath et al., Proc. Natl. Acad. Sci. USA, 84: 7109-7113 (1987); U.S. Pat. No. 5,496,552). However, BMPs have also been detected in or isolated from other mammalian tissues and organ including kidney, liver, lung, brain, muscle, teeth, and gut. BMPs may also be produced using standard in vitro recombinant DNA technology for expression in prokaryotic or eukaryotic cell cultures (see, e.g., Wang et al., Proc. Natl. Acad. Sci. USA, 87: 2220-2224 (1990); Wozney et al., Science, 242: 1528-1534 (1988)). Some BMPs are commercially available for local use as well (e.g., BMP-7 is manufactured and distributed for long bone non-union fractures by Stryker-Biotech (Hopkinton, Mass., U.S.); BMP-2 is manufactured and distributed for long bone acute fractures by Wyeth (Madison, N.J., U.S.), and also for spinal fusions by Medtronic, Inc. (Minneapolis, Minn., U.S.).

BMPs normally exist as dimers of the same monomeric polypeptides (homodimers) held together by hydrophobic interactions and at least one interchain (between monomers) disulfide bond. However, BMPs may also form heterodimers by combining the monomers of different degrees (lengths) of processing (e.g., a full-length, unprocessed monomer associated with a processed, mature monomer) or from different BMPs (e.g., a BMP-6 monomer associated with a BMP-7 monomer). A BMP dimer of unprocessed monomers or a BMP heterodimer of one processed BMP monomer and one unprocessed BMP monomer are typically soluble in aqueous solutions as is a dimer of processed monomers that remain in a non-covalent complex with their corresponding cleaved prodomains (i.e., “soluble BMP”, “sBMP”), whereas a BMP dimer of processed (mature) monomers (that are separated from their corresponding cleaved prodomains) is only soluble in an aqueous solution at a low pH (e.g., acetate buffer, pH 4.5) (see, e.g., Jones et al., Growth Factors, 11: 215-225 (1994)). Both homodimers and heterodimers of BMP-6 and BMP-7 may be used in the methods and compositions described herein.

The BMPs useful in the methods described herein for regulating blood glucose levels in an insulin-independent manner also possess an “osteoinductive” or “osteogenic” activity, i.e., the ability to stimulate bone formation in a standard osteoinductive assay. Such osteoinductive assays include ectopic bone formation assays in which a carrier matrix comprising collagen and a BMP are implanted at an ectopic site in a rodent, and the implant then monitored for bone formation (Sampath and Reddi, Proc. Natl. Acad. Sci. USA, 78: 7599-7603 (1981)). In a variation of such an assay, the matrix may be implanted at an ectopic site and the BMP administered to the site, e.g., by intravenous injection into the rodent. Another way to assay for BMP osteoinductive activity is to incubate cultured fibroblast progenitor cells with a BMP and then monitor the cells for differentiation into chondrocytes and/or osteoblasts (Asahina et al., Exp. Cell. Res. 222: 38-47 (1996)). Both homodimers and heterodimers of BMP-6 and BMP-7 exhibit osteoinductive activity. Particularly preferred BMPs useful in the methods and compositions of the invention are BMP-6, BMP-7, and heterodimers thereof.

By “pharmaceutically acceptable” is meant a material that is not biologically, chemically, or in any other way, incompatible with body chemistry and metabolism and also does not adversely affect the desired, effective activity of a bone morphogenetic protein that may be administered to an individual to promote uptake of serum glucose by peripheral cells and tissues or to treat or prevent diabetes according to the invention.

The terms “disorder” and “disease” are synonymous, and may refer to any pathological condition irrespective of cause or etiological agent.

A “drug” refers to any compound (e.g., a protein, peptide, organic molecule) or composition that has a pharmacological activity. Thus, a “therapeutic drug” is a compound or composition that can be administered to an individual to provide a desired pharmacological activity, e.g., to stimulate uptake of serum glucose by peripheral cells and tissues or to treat a disease, including amelioration of one or more symptoms of a disease. A “prophylactic drug” is a compound or composition that can be administered to an individual to prevent or provide protection from the development in an individual of a disease. A drug may have prophylactic as well as therapeutic uses. For example, treating an individual with a BMP according to the invention promotes uptake of serum glucose by peripheral cells and tissues, which in turn protects the individual from developing a hyperglycemic condition, diabetes, and other complications associated with hyperglycemia and diabetes. Accordingly, unless indicated otherwise, a “treatment” of (or “to treat”) a condition or disease according to the invention comprises administration of a BMP as described herein to an individual to provide therapeutic and/or prophylactic benefits to the individual.

The terms “composition”, “formulation”, “preparation”, and the like are synonymous and refer to a composition that may consist of one or more compounds, e.g., a composition comprising a BMP and a pharmaceutically acceptable carrier.

The terms “oral”, “orally”, enteral”, “enterally”, “non-parenteral”, “non-parenterally”, and the like, refer to a route or mode for administering an effective amount of a compound, such BMP-6, BMP-7, or composition thereof, to an individual anywhere along the alimentary canal of the individual. Examples of such “enteral” routes of administration include, without, limitation, from the mouth, e.g., swallowing a solid (e.g., pill, tablet, capsule) or liquid (e.g., syrup, elixir) composition; sub-lingual (absorption under the tongue); nasojejunal or gastrostomy tubes (into the stomach); intraduodenal administration; and rectal (e.g., using suppositories for release and absorption of a compound or composition in the lower intestinal tract of the alimentary canal). One or more enteral routes of administration may be employed in the invention. Thus, unless a particular type of “oral” formulation described herein is specified or indicated by the context, “oral” formulations are the same as “enteral” formulations and broadly encompass formulations that may be swallowed from the mouth as well as those that permit administration of a BMP anywhere along the alimentary canal.

Terms such as “parenteral” and “parenterally” refer to routes or modes of administration of a compound, such as BMP-6, BMP-7, or composition thereof, to an individual other than along the alimentary canal. Examples of parenteral routes of administration include, without limitation, intravenous (i.v.), intramuscular (i.m.), intra-arterial (i.a.), intraperitoneal (i.p.), subcutaneous (s.c.), transdermal (absorption through the skin or dermal layer), nasal or pulmonary (e.g., via inhalation or nebulization, for absorption through the respiratory mucosa or lungs), direct injections or infusions into body cavities or organs, as well as by implantation of any of a variety of devices into the body that permit active or passive release of a compound or composition into the body.

It is understood that this invention is particularly directed to stimulating uptake of glucose in the blood circulating in a mammal. Unless specifically indicated otherwise, terms such as “serum glucose”, “blood glucose”, “circulating glucose”, and other similar terms are synonymous and may be used interchangeably in describing the invention. Levels of serum glucose are easily measured, e.g., using a sample of venous blood drawn from an individual, by any of a variety of methods available in the art.

The meaning of other terms will be evident by the context of use and, unless otherwise indicated, are consistent with the meanings understood by those skilled in the fields of medicine, pharmacology, and molecular biology.

Therapeutic Methods and Compositions

As shown herein, bone morphogenetic proteins, such as BMP-6, BMP-7, and heterodimers thereof, are able to stimulate or otherwise promote uptake of glucose circulating in the blood (serum glucose) by peripheral cells and tissues of normal as well as diabetic individuals. Accordingly, methods and compositions described herein provide the pharmacological activity that is useful for regulating blood glucose levels and for treating (or preventing) an undesirable hyperglycemic condition as well as diabetes. Moreover, as such BMPs are able to stimulate uptake of blood glucose by peripheral cells and tissues independently of insulin (endogenously or exogenously provided), methods and compositions described herein may be used to treat both of the major forms of diabetes, i.e., type 1 diabetes (also known as insulin-dependent diabetes mellitus, “IDDM”) as well as type 2 diabetes (also known as non-insulin dependent diabetes mellitus, “NIDDM”), and these methods and compositions may be used instead of currently available insulin-based regimens. Accordingly, the invention provides methods of regulating blood glucose levels, of treating hyperglycemia, and of treating type 1 or type 2 diabetes in an individual comprising administering to the individual an effective amount of a BMP (e.g., BMP-6, BMP-7, or heterodimers thereof).

Pancreatitis is a disease in which the pancreas becomes inflamed because its own digestive enzymes, such as amylase, become activated and attack the organ. The acute form of pancreatitis may occur in individuals experiencing hyperglycemia, and chronic pancreatitis may be found in diabetic individuals. As shown herein (see, Example 3), administration of BMPs lowers serum amylase levels during hyperglycemia, indicating that the methods and compositions described herein may be used to treat both acute and chronic forms of pancreatitis as well as be used as an agent to modulate or control exocrine pancreatic function, which may be desirable even in the absence of pancreatitis.

As noted above, the methods and compositions of the invention are effective to regulate blood glucose levels in normal and diabetic individuals independently of endogenously or exogenously supplied insulin, however, it is also an aspect of the invention that a BMP may be administered to an individual in combination with insulin and/or one or more other drugs currently employed in regimens to control levels of blood glucose or to treat type 1 or type 2 diabetes. Use of such combination regimens or periodic use of different regimens to treat the condition of a particular individual will ultimately be at the discretion of the attending healthcare professional.

Administration of compositions comprising BMP to an individual according to the invention may be achieved by intravenous (i.v.) injection of a solution comprising a BMP, or by any other parenteral or oral route that will provide an individual with an effective amount of BMP in the blood to stimulate uptake of blood glucose by peripheral cells and tissues. Various pumps or slow release technologies that provide continuous or intermittent infusions may also be employed to maintain desirable levels of a BMP circulating in the blood of an individual.

While it is possible that a BMP may be administered alone as the raw chemical, it is more likely that a BMP will be administered to an individual as an active ingredient in a pharmaceutical composition. Standard methods of preparing dosage forms are known, or will be apparent, to those skilled in this art (see, e.g., Remington's Pharmaceutical Sciences, 18th edition, (Alfonso R. Gennaro, ed.) (Mack Publishing Co., Easton, Pa. 1990)).

The invention thus further provides a pharmaceutical composition comprising a BMP, or a pharmaceutically acceptable salt thereof, together with one or more pharmaceutically acceptable carriers and, optionally, one or more other therapeutic or beneficial agents, such as, another drug for treating hyperglycemia or diabetes, an antibiotic, an antiviral compound, an anti-fungal drug, a vitamin, a trace metal supplement, or ions to restore or maintain proper ionic balance in blood or other tissues. Such agents may be administered to an individual together with or separately from the BMP. Clearly, the combination therapies described herein are merely exemplary and are not meant to limit possibilities for other combination treatments or co-administration regimens comprising a BMP.

A pharmaceutically acceptable carrier used in a pharmaceutical composition of the invention must be “acceptable” in the sense of being compatible with the physiology of a patient and also non-deleterious to the activity of the BMP or of the beneficial property or activity of any other ingredient that may be present in a composition that is to be administered to a patient.

Pharmaceutical compositions comprising a BMP for use in the invention may include those suitable for administration by a parenteral or enteral (along the alimentary canal) route, including (without limitation), an intravenous (i.v.), subcutaneous (s.c.), oral (swallowing by mouth), sub-lingual (absorption under the tongue), rectal (e.g., suppositories), nasal (e.g., inhalation or insufflation), auricular (ear), ocular, topical, transdermal, or vaginal route.

A pharmaceutical composition may, where appropriate, be conveniently presented in discrete dosage units and may be prepared by any method known in the art. Such methods may include the step of bringing a BMP into association with liquid carriers or finely divided solid carriers or both and then, if necessary, shaping the product into the desired composition.

For example, a BMP may be formulated for parenteral administration and may be presented in unit dose form in ampoules, pre-filled syringes, a small volume infulsion, or in multi-dose containers with, e.g., an added preservative. The compositions may take such forms as suspensions, solutions, or emulsions in oily or aqueous vehicles, and may contain formulation agents such as suspending, stabilizing, and/or dispersing agents. Alternatively, a BMP may be prepared and supplied in a crystallized, lyophilized, or other solid form (e.g., as obtained by aseptic isolation of sterile solid or by lyophilization from solution) for constitution with a suitable aqueous vehicle, e.g., sterile, pyrogen-free water, or sterile physiological buffer, prior to parenteral administration.

Pharmaceutical compositions suitable for oral administration of BMP may conveniently be presented as discrete units such as capsules, cachets, or tablets containing a predetermined amount of a compound of the invention in a powder or granule form, in a solution, in a suspension, or as an emulsion. A formulation comprising a BMP may also be presented as a bolus, electuary, or paste. Tablets and capsules for oral administration may contain conventional excipients such as binding agents, fillers, lubricants, disintegrants, or wetting agents.

Orally administrable, liquid preparations comprising a BMP may be in the form of, by way of example, aqueous or oily suspensions, solutions, emulsions, syrups, or elixirs, or may be presented as a dry product for constitution with water or other suitable vehicle before use. Such liquid preparations may also contain one or more conventional additives, including but not limited to suspending agents, emulsifying agents, non-aqueous vehicles (which may include edible oils), preservatives, and the like.

Other compositions suitable for oral administration of a BMP via the mouth include, without limitation, lozenges comprising BMP, optionally, in a flavored base, and comprising sucrose, acacia, and/or tragacanth; pastilles comprising BMP-7 in an inert base such as gelatin and glycerin or sucrose and acacia; and mouthwashes comprising the active ingredient (BMP) in a suitable liquid carrier.

Pharmaceutical compositions suitable for rectal administration may comprise a BMP and a carrier that provides a solid unit dose suppository. Suitable carriers include cocoa butter and other materials commonly used in the art, where the suppository may be conveniently formed by admixture of BMP with the softened or melted carrier(s) followed by chilling and shaping in molds.

For intra-nasal administration, e.g., administration to the inner nasal surfaces and/or mucous membranes, a composition comprising a BMP may be used as a liquid spray or dispersible powder or in the form of drops. Drops may be formulated with an aqueous or non-aqueous base also comprising one more dispersing agents, solubilizing agents, or suspending agents. Liquid sprays may conveniently be delivered from pressurized packs.

For administration to the lungs by inhalation, a BMP may be delivered from an insufflator, nebulizer, a pressurized pack, or other convenient means known in the art for delivering a protein (e.g., insulin) by inhalation. Pressurized packs may comprise a suitable propellant, such as dichlorodifluoromethane, trichlorofluoromethane, dichlorotetrafluoroethane, carbon dioxide, or other suitable gas. In the case of a pressurized aerosol, the dosage unit may be determined by providing a valve to deliver a metered amount. Alternatively, for administration by inhalation or insufflation, BMP may be incorporated into a dry powder composition, e.g., in combination with a suitable powder base such as lactose or starch. The powder composition may be presented in unit dosage form in, e.g., capsules or cartridges, or, e.g., in gelatin or blister packs from which the powder mixture comprising BMP may be administered with the aid of an inhalator or insufflator.

Methods for Screening for Candidate Compound to Treat Hyerglycemia or Diabetes

The discovery that BMPs can direct uptake of serum glucose by peripheral cells and tissues independently of insulin also provides a basis for in vitro methods of identifying candidate compounds for treating diabetes. It is now appreciated that any compound that induces synthesis of a BMP is also a candidate drug for stimulating uptake of blood glucose by peripheral cells and tissues, for treating hyperglycemia, and/or for treating diabetes in an insulin-independent manner. Any of a variety of methods are known for detecting BMP synthesis, including but not limited to, immunoassays such as enzyme-linked inmuunosorbent asssays (ELISA), BMP-specific mRNA (or cDNA) synthesis assays (e.g., Northern blots, polymerase chain reaction (PCR) assays), and assays for BMPs based on osteoinductive activities as mentioned above (e.g., Sampath and Reddi, Proc. Natl. Acad Sci. USA, 78: 7599-7603 (1981); Asahina et al., Exp. Cell. Res. 222: 38-47 (1996)).

With the goal to identify a candidate compound that is particularly useful for regulating blood glucose, treating hyperglycemia, and/or treating diabetes, particularly preferred is a method that identifies a compound that induces synthesis of BMP in cultures of cells that are more directly involved in blood glucose homeostasis, e.g., pancreatic β-cells, or that are a known major source of BMP circulating in the peripheral blood of an individual, e.g., hepatocytes. Both pancreatic β-cells and hepatocytes are known to possess the necessary genetic information for synthesis of BMPs. Moreover, using standard methods, such cells may also be readily transformed with various recombinant expression vectors available in the art that will direct production of a particular BMP, e.g., BMP-6, BMP-7, or heterodimers thereof.

Accordingly, a particularly preferred method of identifying a candidate compound for use in regulating blood glucose levels, in treating hyperglycemia, or in diabetes may comprise the steps of:
incubating a culture of pancreatic β-cells or hepatocytes in the presence and absence of a test compound, wherein said pancreatic β-cells or hepatocytes comprise functional genetic information necessary for synthesis of a BMP,
assaying said cells for the level of synthesis of the BMP,
comparing the level of synthesis of BMP in the presence and absence of the test compound, wherein a higher level of BMP synthesis in the presence than in the absence of the test compound indicates that the test compound is a candidate compound for treating hyperglycemia or diabetes.

A candidate compound identified by such a method as described above may also be tested in vivo for the ability to decrease the level glucose in the peripheral blood of a mammal, including any of a variety of animal models employed for studying diabetes (see, e.g., Examples 2 and 3, below).

### EXAMPLES

### Example 1

Insulin and glucagon content of pancreatic cells in BMP-6 knock-out and wild type mice.

This study was conducted to determine morphological and histological differences between BMP-6 knock-out (KO) mice and wild type mice as well as to compare the relative incidence (numbers) of insulin and glucagon positive cells present in the pancreases of these animals.

Eight BMP-6 knock-out mice (Solloway et al., Dev. Genet., 22: 321-39 (1998)) and eight wild type mice were sacrificed, and liver, pancreas and duodenum from each mouse was taken for histology. Organs were enclosed in paraformaldehyde, and 5 mm thick sections were subjected to immunohistology. Antibodies used for staining were anti-insulin and anti-glucagon (Sigma, St. Louis, Mo., USA).

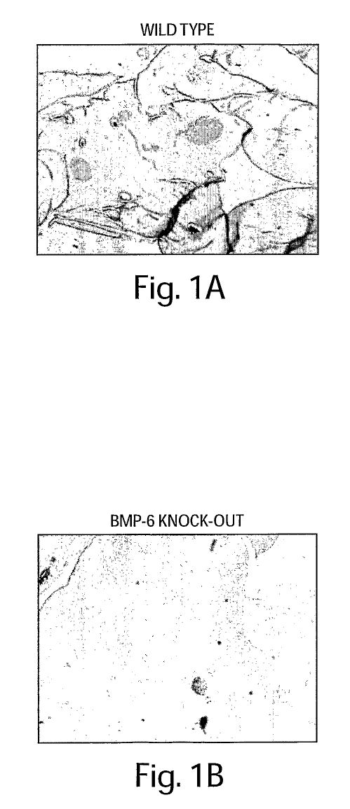

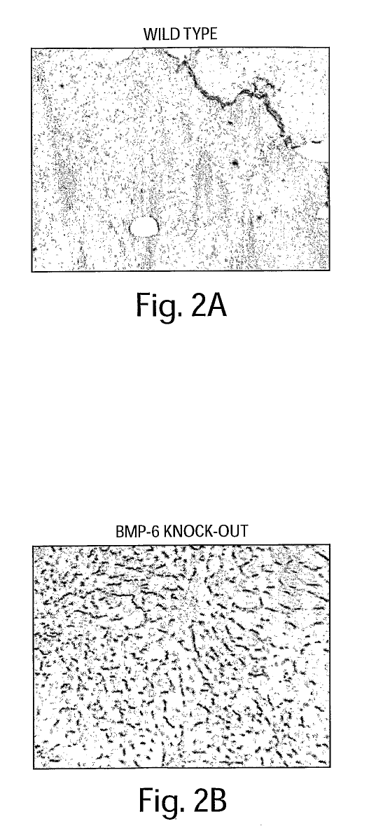

BMP-6 knock-out mice have agenesis of the pancreas and reduction in the size of the stomach and spleen causing fusion of the liver and duodenum. Immunohistochemistry of the pancreas revealed a reduced number of insulin positive cells and Langerhans islands as compared to wild type mice. See, FIGS. 1A (wild type) and 1B (BMP-6 knock-out). Wild type mice had 10±1.4 Langerhans islands per pancreatic section, while BMP-6 knock-out mice had only 1.5±0.7 Langerhans islands per pancreatic section. In addition, immunohistochemistry of livers revealed a clear reduction in the glucagon content of livers from BMP-6 knock-out mice as compared to the livers from the wild type mice. See, FIGS. 2A (wild type) and 2B (BMP-6 knock-out).

The data indicate that BMP-6 knock-out mice have reduced number of Langerhans islands as compared to wild type mice, which should result in a decreased level of insulin and an increased level of blood glucose.

### Example 2

Serum insulin levels in wild type and BMP-6 knock-out mice.

The aim of this study was to determine the serum insulin levels in wild type and BMP-6 knock-out animals and whether any differences in serum insulin levels are correlated with the differences in the number of Langerhans islands in the pancreases of the two groups of mice observed in Example 1, above.

Sera were drawn from 20 wild type animals (wild type controls), from 20 wild type mice 1 hour (h) after intravenous (i.v.) injection of BMP-6 (10 μg/kg of body weight), from 20 BMP-6 knock-out animals (BMP-6 knock-out controls), and from 20 BMP-6 knock-out mice 1 h after injection of BMP-6 (10 μg/kg, i.v.). Levels of insulin in the serum samples were measured by a standard enzyme-linked immunosorbent assay (ELISA) for insulin (Mercodia, Uppsala, Sweden).

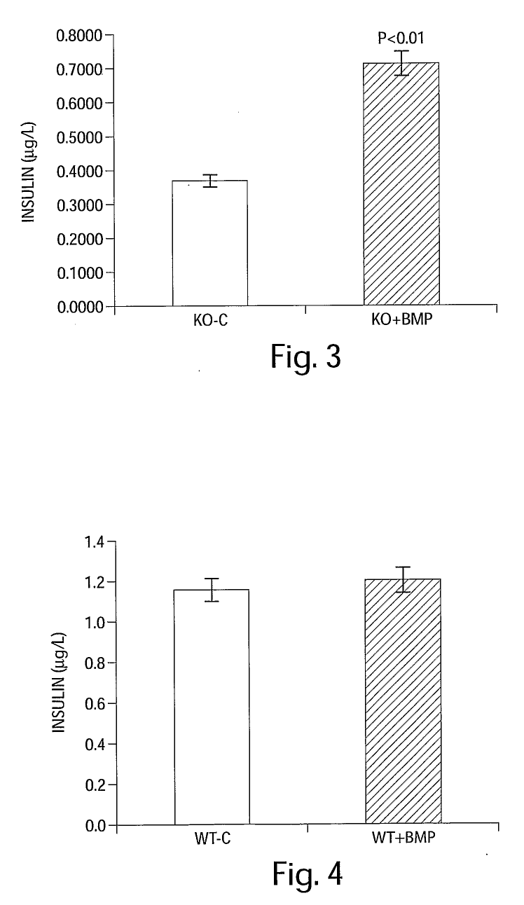

Sera from BMP-6 knock-out control mice (no BMP-6 injection) had reduced levels of serum insulin that were approximately half the levels found in sera from the wild type control animals. However, 1 hour after receiving an i.v. injection of BMP-6, the level of insulin in BMP-6 knock-out mice was elevated two-fold, i.e., comparable to the values measured in wild type control mice. See, FIG. 3. Sera from wild type mice that received an i.v. injection of BMP-6 showed a slight increase in serum insulin levels as compared to mice without the therapy, but the difference was not significant. See, FIG. 4.

These findings show that BMP-6 knock-out mice have reduced levels of serum insulin, i.e., 50% the level in sera of wild type mice, and that this reduced level can be improved by BMP-6 injection in a relatively short amount of time (1 h).

### Example 3

Effects of BMPs on diabetes and exocrine pancreatic function.

This study employed wild type and BMP-6 knock-out animals to determine the effect that intravenous (i.v.) administration of a BMP has on serum glucose levels.

Materials and Methods

Animals and Study Protocol

Three separate animal models were used in this study: one hundred (100) 6 months old Sprague-Dawley female rats, one hundred 3 months old CD-1 female mice, and one hundred 3 months old BMP-6 knock-out female mice. The animals were kept in standard conditions (24° C. and 12 h light/12 h dark cycle) in 20 cm×32 cm×20 cm cages during the experiment. All animals were allowed free access to water and were starved 24 hours before the beginning of the experiment. Each group of animals (rats, wild type mice, and BMP knock-out mice) was divided into the following treatment subgroups:
CONTROL (acetate buffer as a vehicle, i.v.)
BMP-6, 5 μg/kg (of body weight), i.v.
BMP-6, 20 μg/kg, i.v.
BMP-6, 100 μg/kg, i.v.
BMP-6, 300 μg/kg, i.v.
BMP-7, 5 μg/kg, i.v.
BMP-7, 20 μg/kg, i.v.
BMP-7, 100 μg/kg, i.v.
BMP-7, 300 μg/kg, i.v.
sBMP-7, 15 μg/kg, i.v.
sBMP-7, 60 μg/kg, i.v.
sBMP-7 300 μg/kg, i.v.
sBMP-7 900 μg/kg, i.v.
   Animals received a single intravenous (i.v.) injection of either recombinant human mature BMP-6, recombinant human mature BMP-7, or recombinant human soluble BMP-7 (sBMP, containing both mature part and prodomain) at doses of 5 μg/kg (of body weight), 20 μg/kg, 100 μg/kg and 300 μg/kg for mature BMP-6 and BMP-7 and at doses of 15 μg/kg, 60 μg/kg, 300 μg/kg and 900 μg/kg for sBMP-7.
   Glucose Tolerance Test

Immediately after the injection of BMP or vehicle, 2.5 hours following the injection, 4.5 hours following the injection, and 24.5 hours following the injection, animals received glucose in an amount of 650 mg/kg per os (oral delivery). Blood samples were taken prior to the injection and at 45 minutes, 2 hours, 4 hours, 6 hours, and 26 hours following the injection, and approximately 1.5 hours following the oral delivery of glucose.

Biochemical Analyses

Serum levels of glucose, urea, creatinine, phosphate, calcium, aspartate aminotransferase, alanine aminotransferase, lactate dehydrogenase, amylase, lipase, alkaline phosphatase, potassium, and sodium were monitored in all animals throughout the experiment.

Blood glucose was measured using an ACCU-CHECK® glucose assay (Roche, Mannheim, Germany).

Amylase activity was measured using a kinetic spectrophotometric assay as previously described (see, e.g., Bhatia et al., Proc. Natl. Acad. Sci. USA, 95: 4760-4765 (1998); Pierre et al., Clin. Chem., 22: 1219 (1976)). Briefly, plasma samples were incubated with the substrate 4,6-ethylidene (G₇)-p-nitrophenyl (G₁)-1-D-malthoheptoside (Sigma Chemical Co., St. Louis, Mo., USA) for two minutes at 37° C., and the absorbance was measured every minute for the subsequent two minutes at 405 nanometers (um). The change in absorbance was used to calculate amylase activity, expressed as units per liter (“units/L”).

Results

Serum Glucose

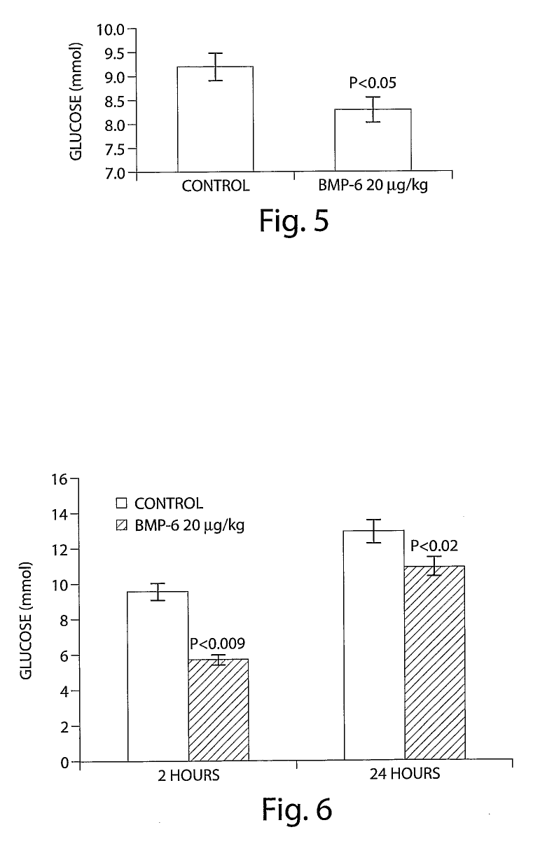

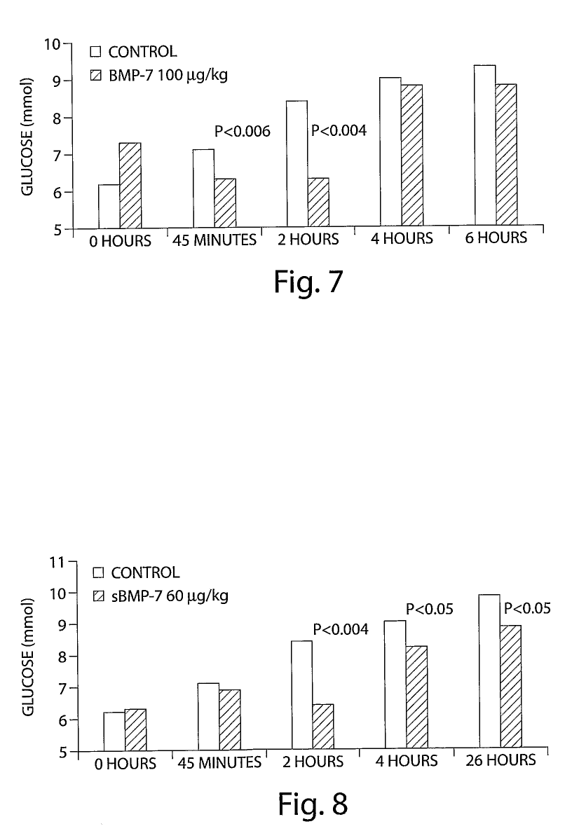

Serum glucose levels were reduced significantly in all groups that received BMP. As shown in FIG. 5 (wild type mice) and FIG. 6 (BMP-6 knock-out mice), both wild type and BMP-6 knock-out mice that received BMP-6 at a dose of 20 μg/kg showed significantly lower serum glucose levels. In particular, levels of glucose in BMP-6 treated wild type mice were reduced to as low as 59.7% of the serum glucose values found in wild type control animals. See, FIG. 5. A significant decrease in blood glucose was also observed in BMP-6 knock-out mice that received BMP-6 (20 μg/kg) compared to control BMP-6 knock-out animals at 2 and 24 hours after receiving BMP-6. See FIG. 6. Rats receiving mature BMP-7 and rats receiving soluble BMP-7 (sBMP-7) had reduced serum glucose levels relative to control animals at 45 minutes and 2 h after the injection of the BMP. See, FIG. 7 (BMP-7) and FIG. 8 (sBMP). The effect was seen until 4 hours following the injection in the case of BMP-7 (FIG. 7) and even at 26 hours in the case of sBMP (FIG. 8).

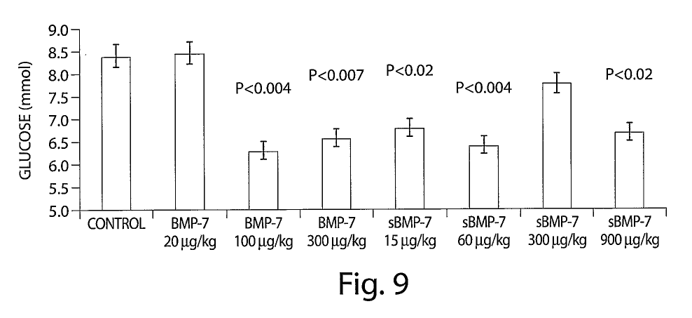

Most of the doses of BMP-7 and sBMP-7 were effective at significantly reducing serum glucose levels in the animals, and an sBMP-7 dose of only 15 μg/kg was equally successful as the higher doses. See, FIG. 9.

Serum Amylase

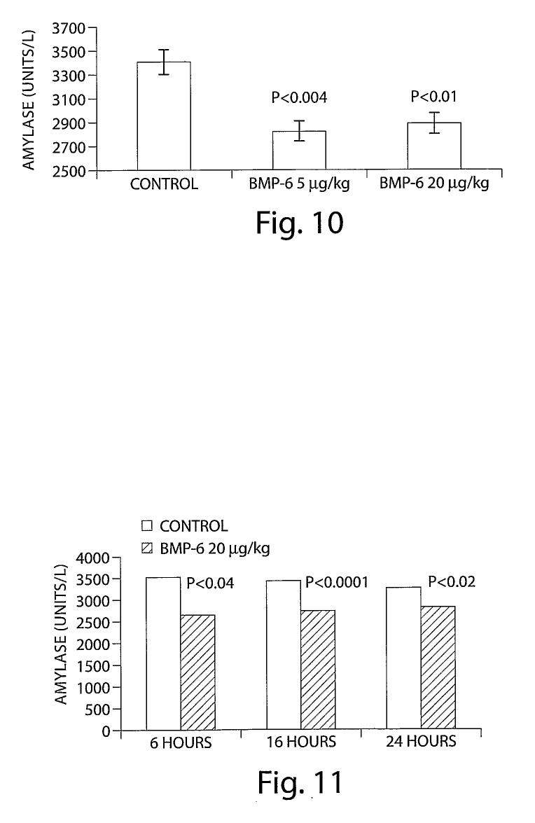

BMP-6 administered at doses of 5 and 20 μg/kg significantly reduced serum amylase levels in wild type mice. See, FIG. 10. Administration of BMP-6 also resulted in a statistically significant lowering of serum amylase values in BMP-6 knock-out mice at various time points measured, e.g., at 6, 16, and 24 hours. See, FIG. 11. Serum amylase level was reduced in BMP-6 treated animals to about 75% the level in control animals.

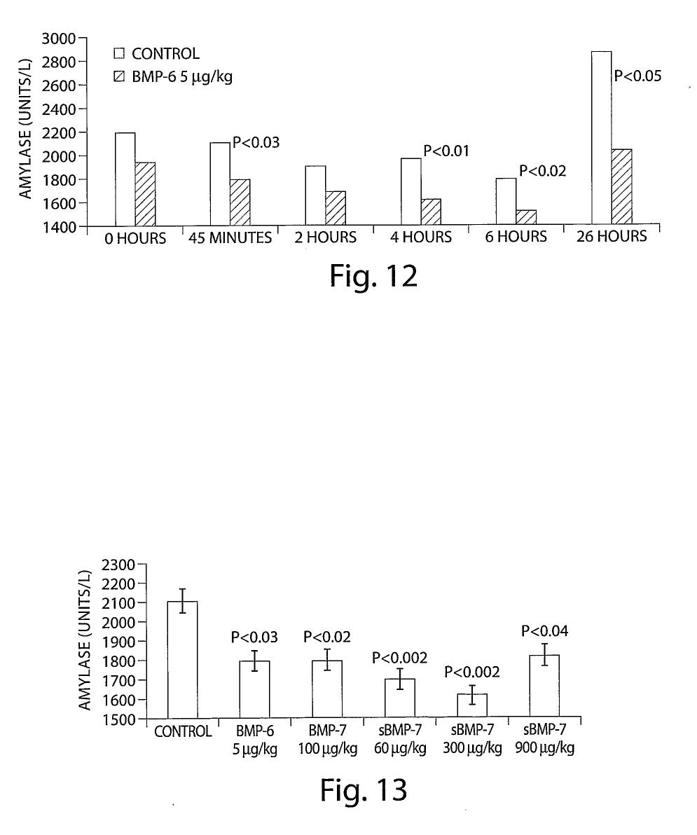

Rats receiving BMP-6 at a dose of 5 μg/kg had significantly lower serum amylase values as compared to control animals at 45 minutes following the injection of BMP and for the duration of the experiment. See, FIG. 12. A comparison of the different doses of BMP-6, BMP-7, and sBMP-7 revealed a similar trend in lowering serum amylase values in comparison to control animals, but no significant differences were observed between individual treatment groups. See, FIG. 13.

Conclusion

BMP-6, BMP-7, and sBMP-7 at different doses significantly reduced serum glucose levels as well as the levels of exocrine pancreatic enzymes, such as amylase. The effect was seen 45 minutes following the application of therapeutic agent (BMP), consistent with a direct, non-genomic mechanism of action. Very small doses, such as 5 and 15 μg/kg, were effective in reducing serum glucose levels. Huge reductions in serum glucose levels, e.g., by more than 40% (see, e.g., FIGS. 5-9) after the treatment with BMPs are of particular interest since diabetes is one of the most dangerous diseases with many problems in current therapy.

Relatively small doses of BMP (e.g., 5 and 15 μg/kg) were also effective at lowering the level of pancreatic amylase activity. The result of a 25% reduction in the serum amylase level has a great potential in treating acute and chronic pancreatitis.

### Example 4

Effect of BMP-6 on ¹⁸fluoro-deoxyglucose (¹⁸FDG).

A preliminary study was conducted to determine the effect of intravenously (i.v.) administered BMP-6 on the level of circulating serum glucose as followed using ¹⁸fluoro-deoxy-glucose (¹⁸FDG).

Animals and Study Protocol

Three 4-months old Sprague-Dawley female rats received ¹⁸fluoro-deoxyglucose via rat tail vein. The rats were divided as follows:
animal 1 received ¹⁸FDG, i.v. only
animal 2 received ¹⁸FDG, i.v., and BMP-6 at 60 μg/kg (of body weight), i.v., at the same time
animal 3 received ¹⁸FDG, i.v., and BMP-6 at a dose of 60 μg/kg, i.v., at 2 h before administration of ¹⁸FDG
   Blood Samples

Blood from the orbital plexus was taken 30, 120, and 180 minutes following the administration of ¹⁸FDG. A sample of 0.5 mL of blood was taken for measurement.

Sacrifice

Animals were sacrificed 180 minutes following the administration of ¹⁸FDG. Blood and all organs were taken for measurement.

Measurement of Radioactivity with Gamma Counter

All samples were measured for the amount of radioactivity with a gamma counter and ¹⁸FDG levels were expressed as counts per minute (cpm). All values were corrected in dependence of the half-life factor.

Results

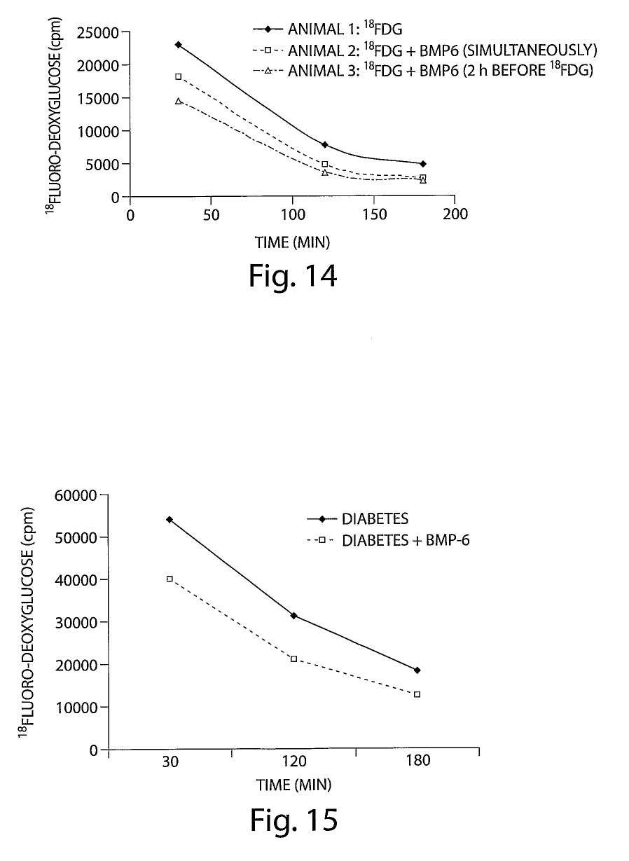

Results of this study are shown graphically in FIG. 14. Animals receiving BMP-6 at a dose of 60 μg/kg, i.v., had reduced ¹⁸FDG blood levels 30 minutes following the administration of ¹⁸FDG. Animal 2 that received BMP-6 at the same time as ¹⁸FDG had a 22% reduction in blood ¹⁸FDG level as compared to control rats. Animal 3 that received BMP-6 2 hours before ¹⁸FDG administration had a 37% reduction in blood ¹⁸FDG level as compared to control rats.

The trend remained throughout the experiment and at 180 minutes following the administration of ¹⁸FDG. Animals that received BMP-6 at the same time as ¹⁸FDG had a 44% reduction of blood ¹⁸FDG levels. Animals that received BMP-6 2 hours before ¹⁸FDG had a 53% reduction of blood ¹⁸FDG levels as compared to control rats.

The data indicate that BMP-6 reduces blood glucose levels up to 53% as compared to control animals at 2 hours following i.v. administration.

### Example 5

Further study of the effect of BMP-6 on ¹⁸FDG in serum of diabetic animals.

Animals and Study Protocol

Four months old Sprague-Dawley female rats received ¹⁸fluoro-deoxyglucose (¹⁸FDG) via rat tail vein. Rats were divided as follows:
Alloxan at a dose of 75 mg/kg (per body weight) to induce diabetic rats receiving ¹⁸FDG, i.v., only
Alloxan (75 mg/kg) to induce diabetic rats receiving ¹⁸FDG, i.v., and 60 μg/kg BMP-6, i.v., at the same time
Normal rats receiving ¹⁸FDG, i.v. only
Normal rats receiving ¹⁸FDG, i.v., and 60 μg/kg BMP-6, i.v., at the same time
   Blood Samples

Blood from the orbital plexus was taken 30, 120, and 180 minutes following the administration ¹⁸FDG. A 0.5 mL sample of blood was taken for measurement

Urine Samples

Urine was collected for 3 hours throughout the experiment.

Sacrifice

Animals were sacrificed 180 minutes following the application of ¹⁸FDG and blood, and all organs were taken for measurement.

Measurement of Radioactivity with Gamma Counter

All samples were measured for the amount of radioactivity with gamma counter and were expressed as cpm (counts per minute). All values were corrected in dependence of the half-life factor.

Results

The alloxan-induced diabetic animals that received BMP-6 at a dose of 60 μg/kg had reduced ¹⁸FDG blood levels at 30 minutes following administration of ¹⁸FDG. As shown in FIG. 15, animals receiving BMP-6 at the same time as ¹⁸FDG had a 26% and a 33% reduction of blood ¹⁸FDG levels at 30 minutes and 120 minutes, respectively, following the administration of ¹⁸FDG as compared to control rats.

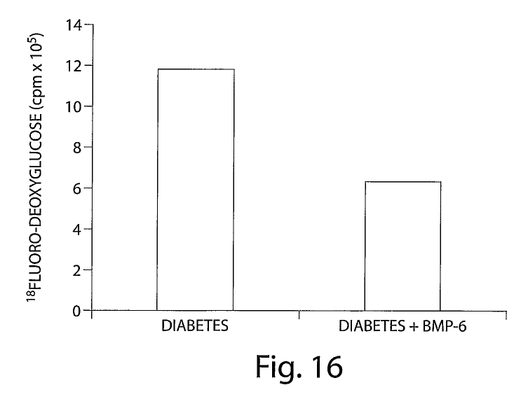

Diabetic animals receiving BMP-6 had a 46.8% reduction of urine ¹⁸FDG throughout 3 hours of experiment. See, FIG. 16. Normal animals receiving BMP-6 had a 12% reduction in blood ¹⁸FDG at 120 minutes following the administration of ¹⁸FDG, and urine ¹⁸FDG was not detectable, suggesting there was no ¹⁸FDG in urine of normal rats (data not shown).

Conclusion

The data confirm the findings of the preliminary study in Example 4, above. BMP-6 reduced blood glucose levels of diabetic animals up to 33% as compared to control diabetic animals at 2 hours following administration of the BMP-6. BMP-6 also reduced ¹⁸FDG urine levels throughout the experiment, suggesting that reduction of blood ¹⁸FDG levels is not the result of increased secretion of ¹⁸FDG, but increased metabolism by peripheral tissues.

### Example 6

BMP-6 reduces blood glucose levels in NOD mice.

The goal of this study was to determine whether BMP-6 can reduce blood glucose levels in severely diabetic NOD mice (Harlan, Indianapolis, Ind., USA) to near normal levels and to determine the length of time that such reduced levels of glucose can be maintained. NOD mice in terminal phase of diabetes are void of insulin production (as in type 1 diabetes) and, thus, require administration of insulin every 12 hours to avoid dying of severe hyperglycemia.

Six (6) NOD mice were injected once with BMP-6 at 60 μg/kg, i.v., while two NOD mice were injected with insulin every 12 hours (h). Blood glucose levels were measured with test strips before the beginning of experiment and then at 0.5, 2, 6, 12, 24, 30, 48, 58, 72, 78, 85, 89, 96, 112, 120, 144, 153, and 168 h following the beginning of experiment.

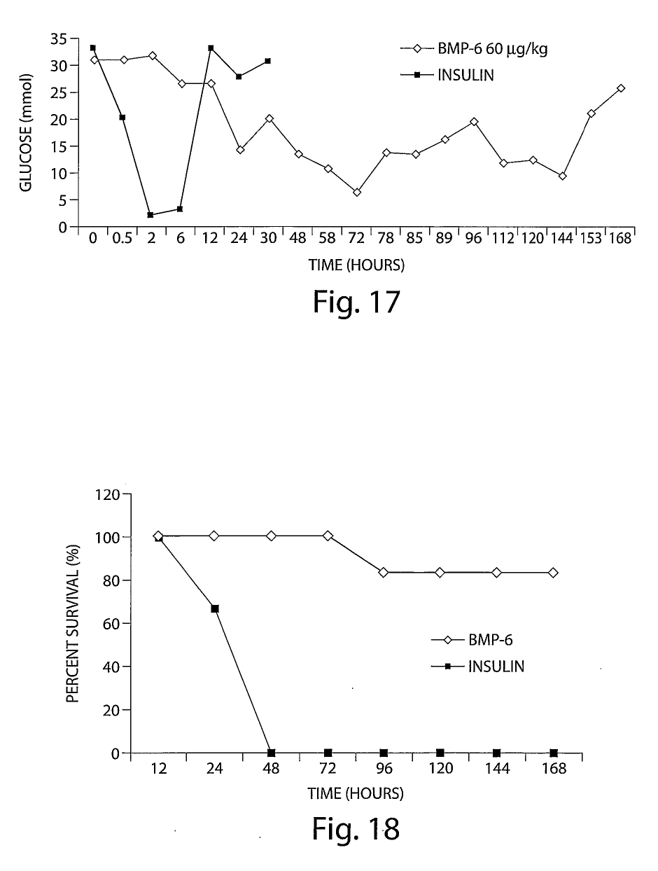

Insulin quickly reduced blood glucose levels in both NOD mice within the period of 2 h, maintained low levels for 4 h, and within 12 h completely lost its effect resulting in extremely high glucose levels (33 mmol). See, FIG. 17. Both animals treated with insulin died within 30 h. The six animals that received one injection of BMP-6 (60 μg/kg, i.v.) had reduction in blood glucose levels at 24 h following the beginning of the experiment and maintained normal glucose levels for 153 h. See, FIG. 17. Furthermore, 5 out of the 6 animals treated with BMP-6 survived the experiment without any insulin supplementation. See, FIG. 18.

Conclusion

A single, intravenous injection of BMP-6 reduced extremely high blood glucose levels of terminally diabetic mice and maintained normal blood glucose levels for 153 h in the absence of any insulin supplementation. The data show that BMP-6 is effective at restoring and maintaining normal glucose levels by an insulin independent pathway or mechanism. Accordingly, since BMPs can regulate blood glucose levels independently of insulin, the data also support the use of BMPs to treat both type 1 diabetes (loss of insulin production) as well as type 2 diabetes (loss of response to insulin).

### Example 7

Mechanism of action of BMPs on diabetes.

The aim of these experiments was to investigate the mechanism of action of BMPs on glucose pathways.

NOD mice (Harlan, Indianapolis, Ind., USA) were injected with BMP-6 at a dose of 60 μ/kg (of body weight) and were sacrificed at different time points: 0 hours (h), 2 h, 6 h, 12 h, 72 h and 7 days.

At the sacrifice, livers were immersed into Trizol RNA isolation reagent (Life Tchnologies, Grand Island, N.Y., USA), and RNA was isolated following the Trizol RNA isolation protocol according to the manufacturer. RNA was later transcribed to cDNA, which was further analyzed by real time polymerase chain reaction (PCR).

Expression of different genes was analyzed using primers to identify transcripts for the following proteins: β-actin, PEPCK, PGC1α, HMG CoA lyase, glucose-6-phosphatase, and acetyl CoA acyltransferase.

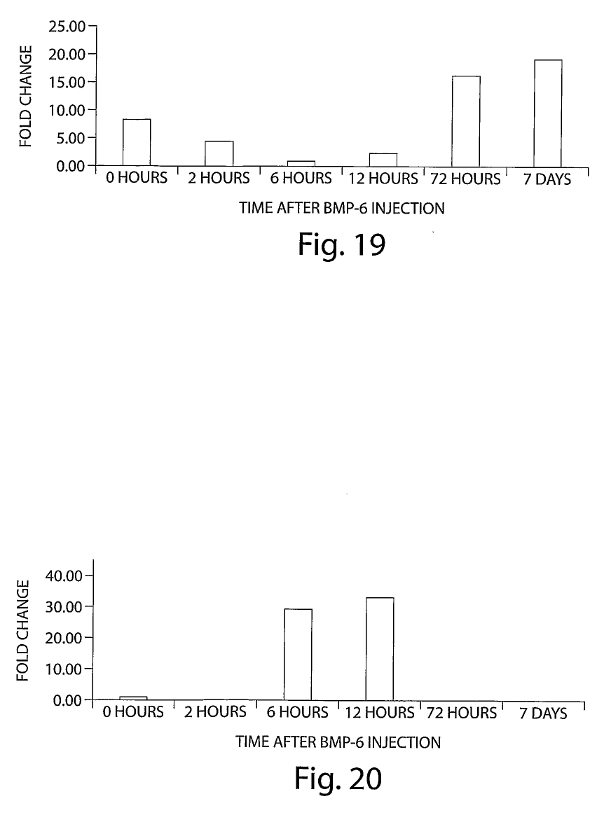

PEPCK is a crucial enzyme involved in the gluconeogenic pathway. PEPCK expression in the liver of NOD mice was reduced 9.6-fold at 6 hours after the injection of BMP-6 as compared with the level of PEPCK expression in mice prior to administration of BMP-6 (0 hours). FIG. 19 shows the fold change in PEPCK levels at various times compared with the level in mice 6 hours after administration of BMP-6 (bar at 6 hours is one-fold change). Expression of PEPCK was reduced throughout the experiment.

PGC1α (“PGC1alpha”) is a mitochondrial transcriptional factor that increases the production of oxidative enzymes. PGC1α expression in liver was increased 30-fold at 12 hours following the injection of BMP-6 compared to the level of expression prior to administration of BMP-6 (0 hours). FIG. 20 shows the fold change in PGC1α levels at various times compared with the level in mice prior to (0 hours) receiving an injection of BMP-6 (bar at 0 hours is one-fold change).

For the other genes, the effect on expression was less pronounced with a maximum of a 3-fold change observed.

HMG CoA lyase catalyzes the last step of ketogenesis. HMG CoA lyase expression was reduced 2.7-fold at 12 hours following administration of BMP-6.

Conclusion

The expression of PEPCK in the liver was reduced 9.6-fold showing reduced gluconeogenesis. The expression of PGC1α in the liver was increased 30-fold suggesting that oxidative metabolism was increased. The observed reduction in hepatic glucose production and the accompanying activation of expression of oxidative metabolism after BMP-6 injection are consistent with a reduction of glycemia via an insulin independent mechanism.

### Example 8

Furin-mediated reduction of blood glucose.

Furin is an endoprotease that processes a variety of proproteins by proteolytic cleavage to release the active form of the proteins. This study was made to determine whether the immature form of BMP that is circulating in the bloodstream could be activated by furin and whether such “in situ activated” BMP would have the same effect as BMP given exogenously.

A total of 36 Sprague Dawley rats were divided into the following treatment groups:
Control (n=6)
BMP-6, 60 μg/kg (of body weight) (n=6)
Furin 10 μL/kg (n=6)
Glucose 2 g/kg (n=6)
Glucose 2 g/kg+BMP-6 60 μg/kg (n=6)
Glucose 2 g/kg+Furin 10 μL/kg (n=6)
   Furin (2000 IU/μL) at a dose of 10 μL/kg was injected through the rat tail vein. Blood glucose was measured with test strips before the beginning of the experiment and then at 15, 30, 45, and 60 minutes following the injection.

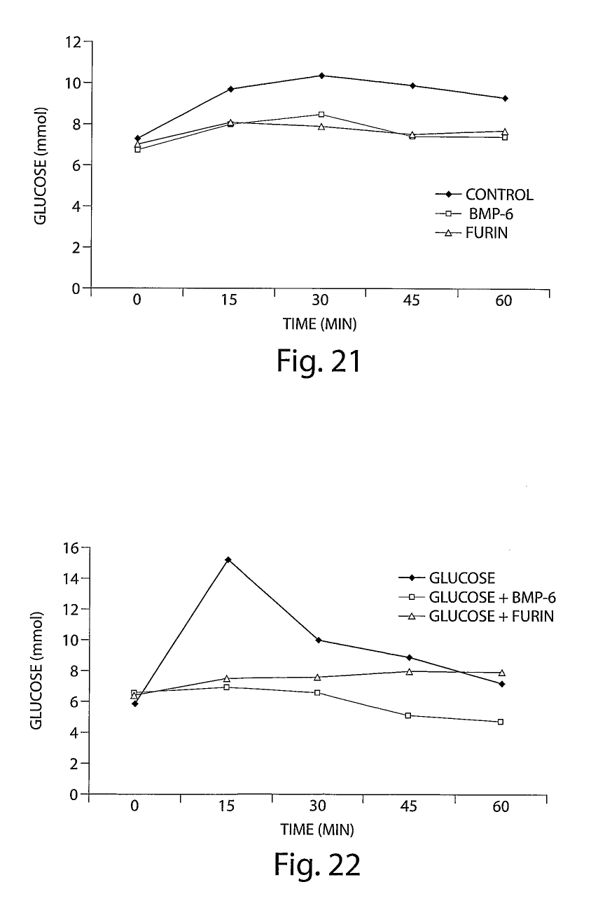

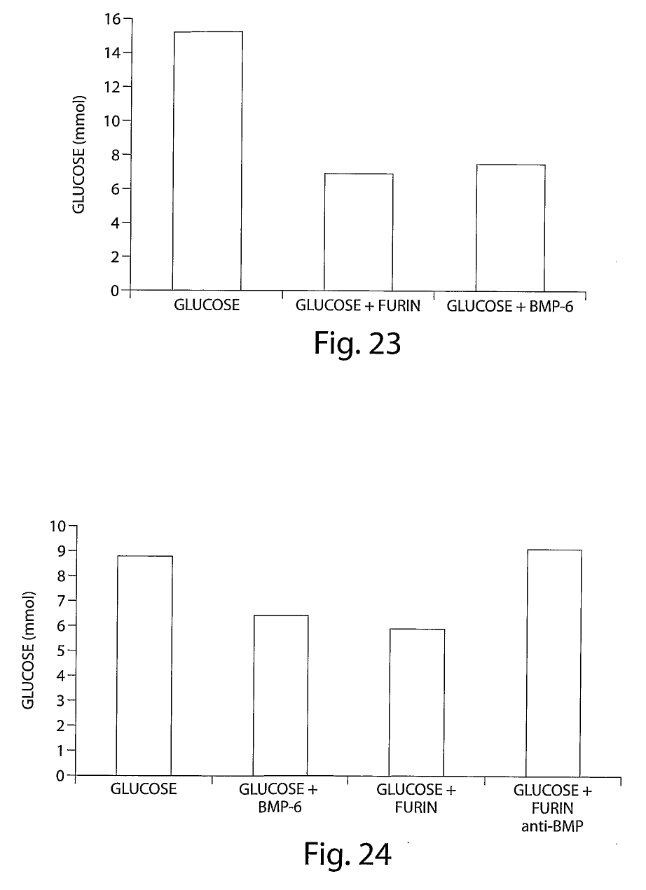

Animals that did not receive glucose did not show significant differences between BMP-6, furin, and control treatment groups, although blood glucose values were lower in BMP-6 and furin treated animals. See, FIG. 21. In contrast, animals that were given a glucose tolerance test by receiving glucose in an amount of 2 g/kg (of body weight), i.v., showed huge differences in blood glucose levels at 15 minutes following the beginning of the experiment. Both BMP-6 and furin significantly reduced serum glucose levels at 15 minutes following the beginning of the experiment and maintained that low value throughout the experiment compared to animals that received glucose alone. See, FIG. 22. At 15 minutes following the beginning of the experiment in animals that received glucose, i.v., furin reduced blood glucose levels by 55%, and BMP-6 reduced glucose levels by 51% as compared to animals receiving only glucose. See, FIG. 23.

Conclusion

Both furin and BMP-6 reduce blood glucose levels suggesting that furin has the same effect as BMP. Furin appears to have activated the immature form of endogenous BMP in the blood.

### Example 9

Further study on furin activation of endogenous BMP.

The aim of this experiment was to determine whether furin acts through BMPs in lowering glucose levels.

Twenty-four (24) animals were divided into the following groups according to the indicated therapy:
Glucose 2 g/kg (of body weight)
Glucose 2 g/kg+BMP-6 60 μg/kg
Glucose 2 g/kg+Furin 10 μL/kg
Glucose 2 g/kg+Furin 10 μL/kg+anti-BMP Polyclonal Antibody
   Results are shown in the bar graphs in FIG. 24. At 30 minutes following the beginning of the experiment, animals that received glucose and BMP-6 or glucose and furin had reduced blood glucose levels compared to animals that received glucose alone. Animals that received glucose and furin in combination with anti-BMP polyclonal antibody (cross-reacting with BMP-6 and BMP-7) had no effect on lowering blood glucose levels, i.e., the levels were kept at the values of control animals.
   Conclusion

The data show that the activity of furin on the level of blood glucose can be blocked by antibody to BMP-6 and BMP-7 consistent with the view that furin reduces blood glucose levels via activating endogenous BMP.

### Example 10

Furin activation of serum BMP.

The aim of this experiment was to determine by immunoblotting (Western blot) whether furin can release BMP in the blood by activating endogenous forms.

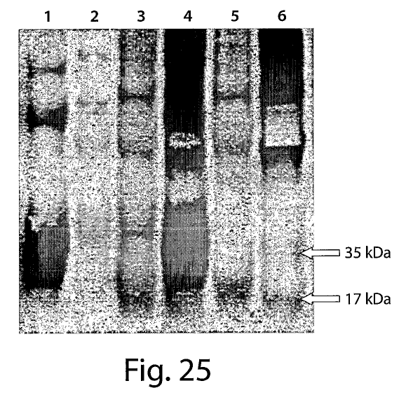

Furin at an amount of 3 μL (2000 IU/μL, New England Biolabs, Beverly, Mass., USA) was added to 0.5 mL of rat plasma. The reaction products were analyzed by Western blotting of gels run under reducing conditions (i.e., with dithiothreitol, “+DTT”) to detect BMP monomer and under non-reducing conditions (i.e., no dithiothreitol, “−DTT”) to detect BMP dimer. Results are shown in the Western blot in FIG. 25. Lanes 1 (−DTT) and 2 (+DTT) of FIG. 25 show BMP-7 standard. Lanes 3 (+DTT) and 4 (−DTT) of FIG. 25 show plasma samples spiked with BMP-7. Lanes 5 (+DTT) and 6 (−DTT) of FIG. 25 show plasma samples with the addition of furin. After adding furin to the plasma, a 35 kilodalton (kDa) band was observed under non-reducing conditions (see arrow, lane 6 of FIG. 25). This 35 kDa species is mature BMP dimer.

Conclusion

Mature BMP band appears on the Western blot of rat plasma treated with furin, suggesting that furin activates endogenous BMP precursor in blood.

All patents, applications, and publications cited in the text above are incorporated herein by reference.

Other variations and embodiments of the invention described herein will now be apparent to those of skill in the art without departing from the disclosure of the invention or the coverage of the claims to follow.

1. A method of stimulating uptake of blood glucose by peripheral cells and tissues in an individual comprising administering to the individual an effective amount of a bone morphogenetic protein (BMP).

2. A method of treating diabetes in an individual by an insulin-independent pathway comprising administering to the individual an effective amount of a bone morphogenetic protein (BMP).

3. The method according to claim 2, wherein said diabetes is type I or type II diabetes.

4. A method of treating hyperglycemia in an individual comprising administering to the individual an effective amount of a BMP.

5. A method of controlling exocrine pancreatic function in an individual comprising the step of administering to the individual an effective amount of a bone morphogenetic protein (BMP).

6. A method of reducing the level of amylase in the blood of an individual comprising administering to said individual an effective amount of a bone morphogenetic protein (BMP).

7. A method of treating pancreatitis in an individual comprising administering to the individual an effective amount of a bone morphogenetic protein (BMP).

8. The method according to any one of claims 1-7, wherein the BMP is selected from the group consisting of BMP-6, BMP-7, and heterodimers thereof.

9. The method according to any one of claims 1-7, wherein the BMP is administered to the individual parenterally.

10. The method according to claim 9, wherein the BMP is administered parenterally by intravenous injection.

11. The method according to any one of claims 1-7, wherein the BMP is administered to the individual in conjunction with or in the absence of insulin.

12. A method of identifying a candidate compound for use in treating diabetes comprising:
incubating cultures of pancreatic β-cells or hepatocytes in the presence and absence of a test compound, wherein said β-cells or hepatocytes comprise functional genetic information necessary for synthesis of a bone morphogenetic protein (BMP),
assaying the cells for the level of synthesis of the BMP,
comparing the level of synthesis of BMP in the presence and absence of the test compound, wherein a higher level of BMP synthesis in the presence than in the absence of the test compound indicates that the test compound is a candidate compound for treating diabetes.

13. The method according to claim 12 further comprising the step of administering the candidate compound to a mammal to determine whether the candidate compound decreases the level of glucose in the serum of the mammal.

14. The method according to claim 12 or 13 further comprising the step of testing the candidate compound in an animal that is a animal model for diabetes to determine whether the candidate compound lowers the level of glucose in the serum of the animal.

15. The method according to claim 14, wherein the animal model is an animal model for type I or type II diabetes.
